(19) United States
(12) Patent Application Publication
(10) Pub. No.: US 2008/0241068 A1
(43) Pub. Date: Oct. 2, 2008

(54) SYSTEM AND METHODS FOR IDENTIFYING INDIVIDUALS AT RISK FOR SYSTEMIC FLUOROPYRIMIDINE TOXICITY

(76) Inventor: James M. Cohen, Ft. Lauderdale, FL (US)

Correspondence Address: BELL, BOYD & LLOYD, LLP, P.O. Box 1135, CHICAGO, IL, 60690, US

(21) Appl. No.: 11/693,340

(22) Filed: Mar. 29, 2007

(51) Int. Cl. A61K 49/00

(52) U.S. Cl. 424/9.2; 424/9.81

(57) ABSTRACT

The present disclosure is related to methods and tests for identifying individuals at risk for systemic toxicity to a substance by administering a topical dose of the substance to the individual and observing any topical response to that substance. In particular, the present disclosure provides methods and tests for determining if an individual is at risk for fluoropyrimidine, such as 5-fluorouracil, toxicity or has a dihydropyrimidine dehydrogenase deficiency by exposing the skin of the individual to a dose of a fluoropyrimidine, such as 5-fluoruracil, using a transdermal skin patch. An inflammatory response of the skin indicates the risk of the individual to such toxicity or enzyme deficiency.

### BACKGROUND

The fluoropyrimidine, 5-fluorouracil (5-FU), was approved by the US Food and Drug Administration for clinical use in 1958. Since then, 5-FU has been used in the treatment of gastrointestinal, breast, and head and neck cancers. According to recent cancer statistics, the previously mentioned cancers are predicted to account for 497,000 new cases and 287,000 deaths in 2007 in the United States alone. Recently, capecitabine (Xeloda®) which is an orally available analog of 5-FU has received FDA approval and is being used to treat gastrointestinal and breast cancers.

5-FU has been found to have an increased rate of response when administered as a long-term continuous infusion as opposed to bolus injections. 5-FU, however, must be metabolized to the nucleotide level in order to exert its efficacious cytotoxic effects on target cells. An anabolic mechanism converts 5-FU into cytotoxic nucleotides responsible for the anti-tumor action of 5-FU. The nucleotide metabolites are also responsible for the toxicity of 5-FU. An alternative catabolic mechanism is responsible for converting up to 80-90% of the 5-FU administered to a patient into biologically inactive metabolites and is controlled by the enzyme dihydropyrimidine dehydrogenase (DPD). Thus, DPD plays a primary role in regulating the amount of 5-FU available to be converted into active metabolite.

5-FU has a relatively narrow therapeutic index. In fact, at least 3-5% of patients treated with these drugs experience severe life-threatening and, at times, lethal toxicity. 5-FU toxicity consists of severe neutropenia, thrombocytopenia, neurological effects, and severe ulceration of the entire gastrointestinal tract. This toxicity is caused primarily but not solely by prolonged excretion of 5-FU and capecitabine. The reason for prolonged excretion appears to be primarily due to deficient levels of DPD in the liver where most of the metabolism of 5-FU occurs. Deficiency in DPD is thought to shift the metabolism of 5-FU toward the anabolic mechanism increasing anabolic metabolites to toxic levels. Accordingly, a deficiency in DPD has been implicated as a cause for sensitivity and toxicity in patients administered 5-FU.

Many patients presenting with severe 5-FU toxicity after administration of standard doses of 5-FU have some form of DPD deficiency. Attempts to develop methods to predict 5-FU toxicity based on DPD deficiency have been published. Examples of tests that could be developed to detect DPD deficiency include pre-treatment liver biopsy analysis of DPD levels. It is thought that pre-treatment liver biopsy analysis of DPD levels is too risky, costly and inappropriate as a screening evaluation to find the 3-5% of patients who could be spared the ravages of 5-FU toxicity.

A substitute for liver biopsy has been developed by determining DPD levels on peripheral blood mononuclear cell preparations (Lu Z, Zhang R, Carpenter J, Diasio R. Decreased DPD Activity in a Population of Patients with Breast Cancer: Implication for 5-FU-Based Chemotherapy, Clinical Cancer Research 1998; 4:325-329), but this test also has a number of problems. In particular, results in large scale studies do not correlate well with 5-FU blood levels. In addition, there is great diurnal variation in levels of DPD activity due to circadian rhythms. Moreover, it appears that as many as 30-40% of patients who develop severe toxicity may be missed using this assay (Mercier C, Ciccoline J. Profiling DPD Deficiency in Patients with Cancer Undergoing 5-FU/Capecitabine Therapy, Clinical Colorectal Cancer 2006; 288-296).

Another method of determining DPD deficiency is genetic testing for genes that code for DPD. The genes which code for DPD have been extensively evaluated for use as screening tests, but there are at least thirty, if not more, mutations and single nucleotide polymorphisms of this gene making it difficult to screen for each polymorphism.

Recently, an assay of the ratio of uracil to dihydrouracil has been used as an indicator of DPD activity. (Ciccolini J, Mercier C, Evrard A, Boyer J, Duffand F, Dahan L, Richard K, Blanquicett C, Milano G, Blesius A, Durand A, Seitz J, Favre R, Lacarelle B. A Rapid and Inexpensive Method for Anticipating Severe Toxicity to Fluorouracil and Fluorouracil-Based Chemotherapy, Theraputic Drug Monitoring 2006; 28:678-685). In this study, 80 patients with severe 5-FU or capecitabine toxicity were studied using this ratio as surrogate of DPD activity. Abnormal values were found in 57 of the 80 patients. 5-FU levels were also measured in some patients, and levels as high as fifteen times higher than the normal range were observed in some patients who failed to become toxic. This suggests that toxicity can occur for reasons other than high levels of drug.

Another attempt to predict 5-FU toxicity in patients has been developed in the form of an assay in which radiolabeled uracil was orally administered and breath analysis of excreted radioactive carbon dioxide correlated with DPD activity. Mattison L, Ezzeldin H, Carpenter M, Modak A, Johnson M, Diasio R. Rapid Identification of DPD Deficiency by Using a Novel 2-13C-Uracil Breath Test. Clinical Cancer Research 2004; 10:2652-2658; and Mattison L, Fourie J, Hirao Y, Koga T, Desmond R, King J, Shimizu T, Diasio R. The Uracil Breath Test in the Assessment of DPD Activity: Pharmokinetic Relationship Between Expired 13C0 2 and Plasma (2-13 C) Dihydrouracil. Clinical Cancer Research 2006; 12:549-554. This assay was based on the fact that DPD is also involved in the metabolism of uracil. This assay has not been used clinically to predict 5-FU toxicity and may also fall short as DPD may not be the only reason for severe 5-FU toxicity. Therefore, no accurate, reproducible, inexpensive, and readily available test is available that addresses safety risks, circadian rhythms leading to poorly correlated DPD levels, multiple genetic polymorphisms, and hitherto undefined reasons for 5-FU toxicity.

### SUMMARY

The present disclosure provides tests and methods for testing an individual for toxicity to a substance. In particular, the present disclosure describes tests and methods including topically administering to an individual a substance, such as fluoropyrimidine, to determine the presence of a reaction of the individual to that substance. In embodiments disclosed herein, the fluoropyrimidine is incorporated into or onto a substrate applied to the skin, such as a transdermal patch, to passively deliver the fluoropyrimidine to the skin of the individual. A topical reaction to the administered fluoropyrimidine indicates the individual is at risk for toxicity to any topically, orally or parenterally administered fluoropyrimidine, or is deficient in an enzyme involved in the metabolism of any fluoropyrimidine.

Accordingly, an embodiment of the present disclosure provides a method of identifying an individual at risk for systemic toxicity of a fluoropyrimidine. The method includes administering an effective amount of the fluoropyrimidine to a portion of skin of the individual and detecting a reaction of the skin to the fluoropyrimidine.

In an embodiment, the fluoropyrimidine is selected from the group consisting of 5-FU, capecitabine, tegafur, tegafur-uracil, floxuridine, 5-fluorodeoxyuridine and S-1.

In an embodiment, the effective amount is between about 3 and about 5 weight percent.

In an embodiment, the substance is administered to the individual through a transdermal patch.

In an embodiment, the method includes administering an amount of an agent sufficient to potentiate the effects of the fluoropyrimidine.

In an embodiment, the agent includes leucovorin.

In an embodiment, the reaction includes at least one of the conditions selected from the group consisting of irritation, swelling and erythema.

Another embodiment of the present disclosure provides a method of detecting a deficiency of an enzyme in an individual. The method includes administering an effective amount of a fluoropyrimidine to a portion of skin of the individual and detecting a reaction of said skin to the fluoropyrimidine.

In an embodiment, the enzyme includes dihydropyrimidine dehydrogenase.

In a further embodiment of the present disclosure, a skin test is provided. The skin test includes an effective amount of fluoropyrimidine on a substrate.

It is therefore an advantage of the embodiments of the present disclosure to provide an accurate, reproducible, inexpensive, non-invasive and safe test to identify an individual at risk for fluoropyrimidine toxicity.

Another advantage of the embodiments of the present disclosure includes providing tests and methods that are not subject to circadian rhythms, multiple genetic polymorphisms or other factors adversely affecting the correlation between the test result and actual toxicity in the individual.

A further advantage of the embodiments of the present disclosure includes providing tests and methods that identify individuals to be treated with a fluoropyrimidine who need a modified dose or alternative therapy.

Additional features and advantages are described herein, and will be apparent from, the following Detailed Description.

### DETAILED DESCRIPTION

The present disclosure describes tests and methods to identify an individual at risk for systemic toxicity of a substance. In particular, the present disclosure is related to topically administering to an individual a substance, such as a fluoropyrimidine, to determine if a topical reaction of the individual to that substance will predict development of systemic toxicity. In embodiments disclosed herein, the substance is incorporated into or onto a substrate applied to the skin, such as a transdermal patch, to deliver the substance to the skin of the individual.

The substance may include any drug or agent which is systemically toxic to an individual when administered systemically at a standard therapeutic dose and which causes a visual change in the appearance of the skin of the individual when applied topically to the skin of the individual.

A substance administered systemically is considered to be administered directly or indirectly into the circulation of an individual and may be administered by topical, enteral or parenteral routes of administration. A substance may be systemically toxic to an individual if it adversely affects the structure, function or life of the individual or system, organ, tissue, cell or sub-cellular component of the individual. Whether a substance is toxic to an individual often depends upon, among other factors, the route of administration of the substance, the duration of exposure of the individual to the substance, the physical state of the substance, the genetic profile of the individual. For example, a substance may be toxic if an enzyme involved in its metabolism is deficient in function or quantity such that the substance is not metabolized into non-toxic metabolites or such that the substance is metabolized by competing mechanisms or enzymes producing toxic levels of the metabolites. Systemic sensitivity or toxicity to a fluoropyrimidine such as 5-FU, for example, may be manifested as conditions such as neutropenia, thrombocytopenia, neurological effects, ulceration of the gastrointestinal tract and even death.

In an embodiment, the substance includes a fluoropyrimidine. A fluoropyrimidine may include, for example, but is not limited to, 5-FU (5-fluoro-1H-pyrimidine-2,4-dione); capecitabine (pentyl N-[1-(3,4-dihydroxy-5-methyl-oxolan-2-yl)-5-fluoro-2-oxo-pyrimidin-4-yl]carbamate); tegafur (5-fluoro-1-(oxolan-2-yl)pyrimidine-2,4-dione; tegafur-uracil (5-fluoro-1-(oxolan-2-yl)pyrimidine-2,4-dione; 1H-pyrimidine-2,4-dione); floxuridine (5-fluoro-1-[(2R,4S,5R)-4-hydroxy-5-(hydroxymethyl)oxolan-2-yl]pyrimidine-2,4-dione); 5-fluoro-1-[(4S,5R)-4-hydroxy-5-(hydroxymethyl)oxolan-2-yl]pyrimidine-2,4-dione; 1-[(2R,3R,4S,5R)-3,4-dihydroxy-5-methyl-oxolan-2-yl]-5-fluoro-pyrimidine-2,4-dione; 5-fluoro-1-[4-hydroxy-5-(hydroxymethyl)oxolan-2-yl]pyrimidine-2,4-dione; 5-fluoro-1-[(4R,5R)-4-hydroxy-5-(hydroxymethyl)oxolan-2-yl]pyrimidine-2,4-dione; [(2R,3 S,5R)-5-(5-fluoro-2,4-dioxo-pyrimidin-1-yl)-3-hydroxy-oxolan-2-yl]methoxyphosphonic acid; S-1 combination (5-chloro-2-hydroxy-1H-pyridin-4-one; 4,6-dioxo-1H-1,3,5-triazine-2-carboxylic acid; and 5-fluoro-1-(oxolan-2-yl)pyrimidine-2,4-dione 5-fluoropyrimidine); or any prodrugs, combinations, derivatives or stereoisomers thereof. In an embodiment, the fluoropyrimidine may include any suitable dihydropyrimidine dehydrogenase inhibitory fluoropyrimidine.

The fluoropyrimidine may be in any suitable form that allows the effective amount of the fluoropyrimidine to be delivered to the skin within a desired period of time. The fluoropyrimidine may be in the form of a liquid, semi-solid, such as a cream, emulsion or gel, or in a solid form.

In embodiments described herein, an effective amount of a fluoropyrimidine is applied to the skin of an individual for a period of time to determine if a visually detectable reaction occurs in that individual. As referred to herein, an effective amount of a fluoropyrimidine is an amount at which the fluoropyrimidine, or metabolite thereof, causes a visible reaction to the skin if the fluoropyrimidine is systemically toxic to the individual. In an embodiment, an effective amount of the fluoropyrimidine is applied to the skin of an individual. The effective amount of the fluoropyrimidine may include from about 2 to about 100, percent by weight of the fluoropyrimidine. The effective amount of the fluoropyrimidine may include from about 3 to about 10 percent by weight of the fluoropyrimidine. The effective amount of the fluoropyrimidine may include from about 3 to about 5 percent by weight of the fluoropyrimidine. The effective amount may include from about 10 to about 100 mg of the fluoropyrimidine or from about 30 to about 50 mg of the fluoropyrimidine.

As referred to herein, topical administration of the fluoropyrimidine refers to contacting the surface of a portion of the skin of an individual with the fluoropyrimidine. The fluoropyrimidine may be applied directly to the skin or, alternatively, delivered to the skin by a substrate encapsulating the fluoropyrimidine in whole or in part. The substrate may include any suitable structure and material that is capable of delivering a fluoropyrimidine to the skin of an individual. In an embodiment, the substrate is in the form of a transdermal patch or skin patch. The transdermal patch may include any suitable transdermal patch known in the art. For example, the patch may be in the form of a laminate structure having a substantially impermeable backing layer, a dispensing membrane containing the fluoropyrimidine to be delivered to the skin and a removable substantially impermeable blocking layer.

The backing layer may serve as a protective cover and structural support while preventing release of fluoropyrimidine from the patch. Examples of materials that may be used to make the backing layer include high and low density polyethylene, polypropylenes, polyvinyl chlorides, polyethylene terephthalates and mixtures thereof.

In an embodiment, the transdermal patch is a self-adhering patch that is designed to be temporarily affixed onto the skin of a patient. The transdermal patch may include any suitable adhesive to hold the substrate in place on the skin. Such materials can be selected from pharmaceutically acceptable adhesive materials such as isobutylenes, silicones, or acrylate adhesives. Representative adhesives include polyisobutylene, silicone adhesives such as silastic, Dow Corning X7-2920 silicone adhesive or Dow Corning 2675 silicone adhesive with or without added silicone-oil tackifiers and solvent-based or water-based acrylate materials.

The fluoropyrimidine may be incorporated into a dispensing membrane of the transdermal patch. The dispensing membrane may include a reservoir, a rate-controlling membrane, a matrix, an adhesive formulation or any combination thereof adapted to release the fluoropyrimidine to the skin of an individual at a controlled rate over a defined period of time.

In certain embodiments the transdermal patch may include other layers involved in regulating the rate of delivery of the fluoropyrimidine to the skin such as a diffusion membrane layer or a pressure sensitive adhesive layer. In such structures, the dispensing membrane may be contained substantially between the backing layer and the diffusion membrane layer or pressure sensitive adhesive layer.

The substantially impermeable blocking layer may be directly affixed to the layer designed to be in contact with the skin. Also, the blocking layer may be removably affixed to the transdermal patch and act to prevent the premature release of the fluoropyrimidine from the patch prior to the application of the patch to the skin of the individual.

A substrate, such as a transdermal patch, may be chosen as a vehicle for delivery of an effective amount of the fluoropyrimidine based on the rate at which the substrate delivers the fluoropyrimidine to the skin of the individual. For example, the transdermal patch may be chosen to have a rate of release of about 10% to about 30% of the dose per day. Other factors may include adherence to the skin for a suitable time period, the absence of reactivity of the skin of the individual to the substrate itself, comfort of the individual or any other suitable property of the substrate that affects the function of the substrate as described herein.

In an embodiment, at least one transdermal patch containing an effective amount of a fluoropyrimidine is applied to the skin of an individual to be tested. The patch may be adhered to the skin of the individual for a time period of between about twelve hours to about one week, or until a diagnostic reaction occurs. It should be appreciated that the time period for a diagnostic reaction to occur for a particular dose of a fluoropyrimidine may vary among different individuals.

In embodiments of the present disclosure, other agents may be combined with the fluoropyrimidine to be tested in order to potentiate the effects of the fluoropyrimidine. Such agents may lower the dose required for the fluoropyrimidine to produce the same reaction as the reaction that would have been produced at a higher dose of the fluoropyrimidine. For example, a folate compound such as leucovorin, a form of folic acid, may be combined with the fluoropyrimidine to lower the dose required to produce a topical reaction indicative of a sensitivity of an individual to systemic fluoropyrimidine. If, for example, the fluoropyrimidine in the aforementioned or higher concentrations do not elicit a measurable inflammatory response, leucovorin may be added to the fluoropyrimidine to potentiate the response. In an embodiment, the dose of leucovorin is equal to the dose of the fluoropyrimidine. In an embodiment, leucovorin is provided at a ratio of 2:1 to about 1:2 of the dose of the fluoropyrimidine or 50-200 percent of the concentration of the fluoropyrimidine. Alternatively, leucovorin may be combined with the fluoropyrimidine in an amount of between about 2 and about 98 weight percent of leucovorin.

A diagnostic reaction or response in the form of a topical reaction or reaction of the skin or a change in the appearance of the skin may include any condition such as conditions associated with an inflammatory reaction or any other visually detectable response. Such conditions may include erythema, swelling, itching or irritation. The reaction to the topically administered fluoropyrimidine may be measured by any suitable method such as by determining a radius of visual or palpable change in the skin from the point of application of the fluoropyrimidine to the skin. For example, in an embodiment employing a transdermal patch to administer the fluoropyrimidine to the skin of the individual, the backing layer of the patch may include a substantially clear plastic portion through which changes in the underlying skin may be visually detected. In addition, the backing layer may include a series of concentric rings surrounding a circular dispensing membrane containing the fluoropyrimidine. The concentric rings may be equally spaced apart and may be marked in color or other indicia to enable a rapid quantification of the degree of erythema or other condition that develops from exposure to the fluoropyrimidine. It should be appreciated that, a diagnostic reaction may indicate toxicity to fluoropyrimidines other than the fluoropyrimidine used to test the individual.

The ability of the embodiments of the present disclosure to elicit a local limited reaction in the skin enables identification of patients who are at risk for a mild to severe, possibly fatal, systemic toxicity when a fluoropyrimidine, such as 5-FU, is administered topically, parenterally or orally to an individual. The measurement of a skin reaction is tantamount to measuring an end-organ response to the fluoropyrimidines. Measuring an end-organ response avoids the limitations of known tests for fluoropyrimidine toxicity caused by circadian rhythms, poorly correlated mononuclear cell DPD levels, genetic polymorphisms, and hitherto undefined reasons for fluoropyrimidine toxicity.

By way of example and not limitation, examples of the present invention will now be given.

### EXAMPLES

### Example I Determining an Effective Amount of 5-FU

A dose of 5-FU used to identify those at risk for fluoropyrimidine toxicity is determined by testing two groups of individuals. The individuals are selected from those who have previously received 5-FU or other fluoropyrimidine. The first group consists of individuals who tolerated treatment with a fluoropyrimidine. The second group consists of individuals identified as having moderate to severe toxicity to a fluoropyrimidine. Transdermal patches containing varying amounts of 5-FU are applied initially to the individuals with good tolerance to determine the highest dose of 5-FU that can be administered without causing a reaction. The doses used are 0 (control), 2, 5, 10, 20, 40, 80 and 100 percent by weight 5-FU. The second group is similarly tested to determine the lowest dose that identifies almost all of the patients at risk. The second group is tested beginning with the lower doses until an identifiable diagnostic response, such as inflammation, occurs. This second group is carefully monitored on a daily basis to avoid precipitation of systemic toxicity from untoward absorption through the skin. Because of the small size of the patch, however, significant systemic absorption is unlikely.

The maximum amount of drug in the patch that does not elicit more than a minimal diagnostic response in a high proportion of individuals in the second group determines an effective amount of 5-F U to be used to identify individuals at risk for fluoropyrimidine toxicity and/or individuals with an enzyme deficiency. In addition, these initial experiments determine how long the patch must remain affixed to the individual to identify individuals at risk for fluoropyrimidine toxicity and/or individuals with an enzyme deficiency. The first group of those individuals who demonstrated good tolerance to previous treatment with the drug should have few to no responses.

### Example II Determining Risk of 5-FU Toxicity

A transdermal patch containing the dose determined in Example I to be an effective amount of 5-FU is applied to the skin of any suitable portion of the body, such as the arm, of an individual to be administered a fluoropyrimidine systemically. In addition, a control patch with no 5-FU may be placed on the individual as a control. The patch remains affixed to the individual for a minimum of one day.

At increments of at least about every twenty-four hours, the skin around each patch is examined to determine the presence of any reaction to the 5-FU such as erythema, swelling or irritation. If a reaction is detected, the extent of the reaction is measured by how many concentric rings are traversed by the changes in the skin.

The presence of a topical reaction to 5-FU and a determination of the extent of the reaction are used to identify individuals at risk for fluoropyrimidine toxicity and to indicate a need to modify the dose of the fluoropyrimidine or to provide an alternative therapy to these individuals.

Embodiments described in the present disclosure may include a skin test. The skin test may be provided in the form of a kit and may include any suitable substrate, such as a transdermal patch, and an effective amount of fluoropyrimidine to be administered to a portion of the skin of an individual as described herein. The skin test may be provided in any suitable container or packaging.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of identifying an individual at risk for systemic toxicity of at least one fluoropyrimidine, said method comprising:
(a.) administering an effective amount of at least one fluoropyrimidine to a portion of skin of the individual; and
(b.) detecting a reaction of the skin to the fluoropyrimidine,
wherein a reaction of the skin to the fluoropyrimidine identifies an individual at risk for systemic toxicity of the fluoropyrimidine.

2. The method of claim 1, wherein the fluoropyrimidine is selected from the group consisting of 5-FU, capecitabine, tegafur, tegafur-uracil, floxuridine, 5-fluorodeoxyuridine and S-1.

3. The method of claim 1, wherein the effective amount of the fluoropyrimidine is between about 2 and about 10 weight percent.

4. The method of claim 1, wherein the fluoropyrimidine is administered to the individual through a transdermal patch.

5. The method of claim 1, which includes administering an amount of an agent sufficient to potentiate the effects of the fluoropyrimidine.

6. The method of claim 5, wherein the agent includes leucovorin.

7. The method of claim 1, wherein the skin reaction includes at least one of the conditions selected from the group consisting of irritation, swelling and erythema.

8. A method of detecting a deficiency of an enzyme in an individual comprising administering an effective amount of a fluoropyrimidine to a portion of skin of the individual and detecting a reaction of the skin to the fluoropyrimidine.

9. The method of claim 8, wherein the enzyme includes dihydropyrimidine dehydrogenase.

10. The method of claim 8, wherein the fluoropyrimidine is selected from the group consisting of 5-FU, capecitabine, tegafur, tegafur-uracil, floxuridine, 5-fluorodeoxyuridine and S-1.

11. The method of claim 8, wherein the effective amount of the fluoropyrimidine is between about 2 and about 10 weight percent.

12. The method of claim 8, which includes administering an amount of an agent sufficient to potentiate the effects of the fluoropyrimidine.

13. The method of claim 12, wherein the agent includes leucovorin.

14. The method of claim 8, wherein the fluoropyrimidine is administered to the individual through a transdermal patch.

15. The method of claim 8, wherein the reaction includes at least one of the conditions selected from the group consisting of irritation, swelling and erythema.

16. A skin test comprising an effective amount of fluoropyrimidine on a substrate.

17. The skin test of claim 16, wherein the fluoropyrimidine is selected from the group consisting of 5-FU, capecitabine, tegafur, tegafur-uracil, floxuridine, 5-fluorodeoxyuridine and S-1.

18. The skin test of claim 16, wherein the effective amount of the fluoropyrimidine is between about 2 and about 10 weight percent.

19. The skin test of claim 16, wherein the substrate includes a transdermal patch.

20. The skin test of claim 16, wherein the topical reaction includes at least one of the conditions selected from the group consisting of irritation, swelling and erythema.
